(19) United States
(12) Patent Application Publication
(10) Pub. No.: US 2022/0301580 A1
(43) Pub. Date: Sep. 22, 2022

(54) LEARNING APPARATUS, ESTIMATION APPARATUS, METHODS AND PROGRAMS FOR THE SAME

(71) Applicant: NIPPON TELEGRAPH AND TELEPHONE CORPORATION, Tokyo (JP)

(72) Inventors: Junji WATANABE, Tokyo (JP); Aiko MURATA, Tokyo (JP)

(73) Assignee: NIPPON TELEGRAPH AND TELEPHONE CORPORATION, Tokyo (JP)

(21) Appl. No.: 17/633,153

(22) PCT Filed: Aug. 6, 2019
(86) PCT No.: PCT/JP2019/030864

§ 371 Date: Feb. 4, 2022

(51) Int. Cl. G10L 25/63; A61B 5/16

(52) U.S. Cl. CPC G10L 25/63; A61B 5/165

(57) ABSTRACT

A learning apparatus includes: a storage unit that stores at least a learning-purpose expression indicating a psychological state and an emotion, and learning-purpose feeling information indicating a feeling at the time of issuance of the learning-purpose expression indicating the psychological state and the emotion; and a learning unit that learns an estimation model, using a plurality of pieces of learning data, one piece of the learning data being a set including at least a chronological series of two or more expressions indicating psychological states and. emotions up to time (t) and learning-purpose feeling information indicating a feeling after the time (t), the estimation model estimating a feeling after a certain time by using at least a chronological series of two or more expressions indicating psychological states and emotions up to the certain time as an input. An estimation apparatus includes an estimation unit that uses at least a chronological series of two or more expressions indicating psychological states and emotions up to a certain time as an input, and with use of an estimation. model that estimates a feeling after the certain time, estimates a future feeling of an object person based on at least input two or more expressions indicating psychological states and emotions of the object person and an. input order thereof.

### TECHNICAL FIELD

The present invention relates to a technique to estimate a feeling from expressions indicating psychological states and emotions, including onomatopoeias.

### BACKGROUND ART

In NPL 1, the impression of an entire onomatopoeia is quantified using a model that predicts the impression of an onomatopoeia from phonological elements, such as the types of consonants and vowels and whether there is dakuon, that compose the onomatopoeia.

### CITATION LIST

### Non Patent Literature

[NPL 1]Yuichiro Shimizu, Ryuichi Doizaki, and Maki Sakamoto, “System That Estimates Subtle Impression of Each Onomatopoeia”, Journal of the Japanese Society for Artificial Intelligence, Vol. 29, No. 1, p. 41-52, 2014.

### SUMMARY OF THE INVENTION

### Technical Problem

The conventional technique estimates the impression evoked by an onomatopoeia, but does not estimate the feeling of a user who uses the onomatopoeia. When it is assumed that the impression of an onomatopoeia matches the feeling of a user of the onomatopoeia, the feeling at the time of the use of the onomatopoeia can be estimated from the used onomatopoeia. However, even in this case, the future feeling after the use of the onomatopoeia cannot be estimated.

An object of the present invention is to provide an estimation apparatus that estimates a future feeling based on expressions indicating psychological states and emotions up to the present time, a learning apparatus that learns a model used in estimating a feeling, methods therefor, and a program.

Note that an expression indicating a psychological state and an emotion represents a psychological state of an oblect person at a certain time point, and is, for example, a general term for a word that is categorized as at least one of an onomatopoeia and an interjection. Furthermore, an onomatopoeia is, for example, a general term for a word that is categorized as at least one of an inanimate phonomime, a pheriomime, and a psychomime. Here, an inanimate phonomime depicts an actual sound using a speech sound, a phenomime depicts a non-auditory sense using a speech sound, and a psychomime depicts a psychological state using a speech sound. Note that an interjection may be referred to as an exclamation. Although the following describes a case where an expression indicating a psychological state and an emotion is an onomatopoeia, processing can be performed similarly also in a case where an expression indicating a psychological state and an emotion is an interjection.

Furthermore, the “feeling”mentioned herein denotes a “mood”, and means the state of a sentiment expressed as “spirited (energetic) or not spirited (not energetic)”, “comfortable or uncomfortable”, “nervous or relaxed”, “relieved or worried”, “positive or negative”, “satisfied or dissatisfied”, “calm or restless”, oy, sadness, anger, and so forth.

### Means for Solving the Problem

In order to solve the aforementioned problem, according to one aspect of the present invention, a learning apparatus includes: a storage unit that stores at least a learning-purpose expression indicating a psychological state and an emotion, and learning-purpose feeling information indicating a feeling at the time of issuance of the learning-purpose expression indicating the psychological state and the emotion; and a learning unit that learns an estimation model, using a plurality of pieces of learning data, one piece of the learning data being a set including at least a chronological series of two or more expressions indicating psychological states and emotions up to time (t) and learning-purpose feeling information indicating a feeling after the time (t), the estimation model estimating a feeling after a certain time by using at least a chronological series of two or more expressions indicating psychological states and emotions up to the certain time as an input.

In order to solve the aforementioned problem, according to another aspect of the present invention, an estimation apparatus includes an estimation unit that uses at least a chronological series of two or more expressions indicating psychological states and emotions up to a certain time as an input, and with use of an estimation model that estimates a feeling after the certain time, estimates a future feeling of an object person based on at least input two or more expressions indicating psychological states and emotions of the object person and an input order thereof.

### Effects of the Invention

The present invention achieves the advantageous effect whereby a future feeling can be estimated based on expressions indicating psychological states and emotions up to the present time.

### BRIEF DESCRIPTION OF DRAWINGS

FIG. 1 is a diagram showing an exemplary configuration of an estimation system according to a first embodiment according to a first embodiment.

FIG. 2 is a diagram for describing an estimation model.

FIG. 3 is a functional block diagram of a learning apparatus according to the first embodiment.

FIG. 4 is a diagram showing an example of a processing flow of the learning apparatus according to the first embodiment.

FIG. 5 is a diagram showing an example of data stored in a storage unit.

FIG. 6 is a functional block diagram of an estimation apparatus according to the first embodiment.

FIG. 7 is a diagram showing an example of a processing flow of the estimation apparatus according to the first embodiment.

FIG. 8 is a diagram showing an example of data stored in the storage unit.

FIG. 9 is a diagram showing an exemplary configuration of a computer that functions as the learning apparatus or the estimation apparatus.

### DESCRIPTION OF EMBODIMENTS

The following describes an embodiment of the present invention. Note: that the constituents that have the same functions or the steps that execute the same processing are given the same reference signs in the drawings used in the following description, and duplicate explanations are omitted. In the following description, it is assumed that processing that is performed for each element of a vector or a matrix is applied to every element of that vector or that matrix, unless specifically stated otherwise.

### First Embodiment

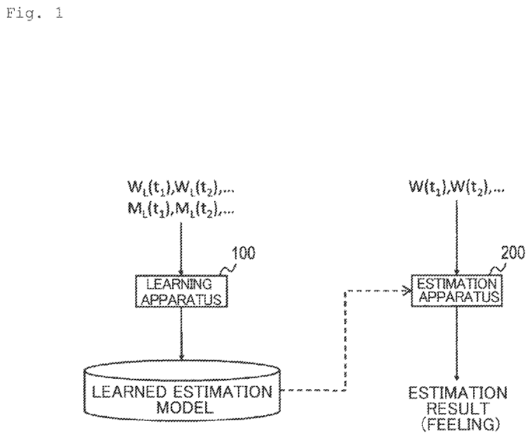

FIG. 1 shows an exemplary configuration of an estimation system according to a first embodiment.

The estimation system of the present embodiment includes a learning apparatus 100 and an estimation apparatus 200.

The learning apparatus 100 learns an estimation model using learning-purpose expressions indicating psychological states and emotions W_{L} (t₁), W_{L} (t₂), . . . and learning-purpose feeling information M_{L} (t₁), M_{L} (t₂), as inputs, and outputs the learned estimation model.

Prior to estimation, the estimation apparatus 200 receives the learned estimation model output from the learning apparatus 100. With use of the estimation model, the estimation apparatus 200 estimates a future feeling using a chronological sequence of estimation-target expressions indicating psychological states and emotions W (t₁), W (t₂), . . . as inputs, and outputs the estimation result. Note that t₁, t₂, . . . are indexes indicating the input order; for example, W(tᵢ) denotes the i^{th} expression indicating a psychological state and an emotion that has been input.

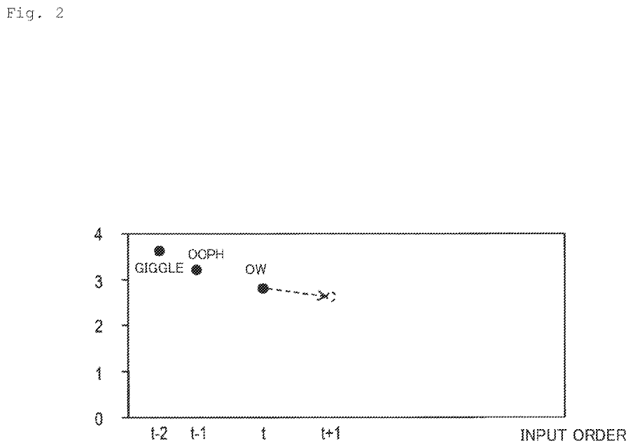

The present embodiment is based on the presumption that, because a feeling goes through temporal changes while maintaining a connection and there is also a connection between an expression indicating a psychological state and an emotion that was issued at a certain time point and a feeling au that time point, the use of these connections makes it possible to estimate, from a chronological series of expressions indicating psychological states and emotions that were input by certain time, a feeling after that time. For example, in a case where the (t−1)^{th} onomatopoeia. is “ooph.”and the t^{th} onomatopoeia is “ow”as shown in FIG. 2, it is estimated that the numerical values of certain feeling information show a decreasing tendency, and the numerical value of the (t+1)^{th} feeling information will become smaller than the numerical value of the i^{th} feeling information. Note that FIG. 2 is an example, and there is a case where the value is estimated to increase in an actual estimation model. Note that feeling information will be described later.

The learning apparatus and the estimation apparatus are, for example, special apparatuses composed of a known or dedicated computer which includes a central computational processing apparatus (CPU: Central Processing Unit), a main storage apparatus (RAM: Random Access Memory), and the like, and which has read a special program. The learning apparatus and the estimation apparatus execute various types of processing under control of the central computational processing apparatus, for example. Data input to the learning apparatus and the estimation apparatus and data obtained through various types of processing are, for example, stored in the main storage apparatus; data stored in the main storage apparatus is read out by the central computational processing apparatus and used in other processing as necessary. Each processing unit of the learning apparatus and the estimation apparatus may be, at least partially, composed of hardware, such as an integrated circuit. Each storage unit included in the learning apparatus and the estimation apparatus can be composed of, for example, a main storage apparatus, such as a RAM (Random Access Memory), or middleware, such as a relational database and a key-value store. Note that each storage unit need not necessarily be included inside the learning apparatus and the estimation apparatus; it is possible to adopt a configuration in which each storage unit is composed of an auxiliary storage apparatus composed of a hard disk, an optical disc, or a semiconductor memory element, such as a Flash Memory, and is provided outside the learning apparatus and the estimation apparatus.

First, the learning apparatus will be described.

<Learning Apparatus 100>

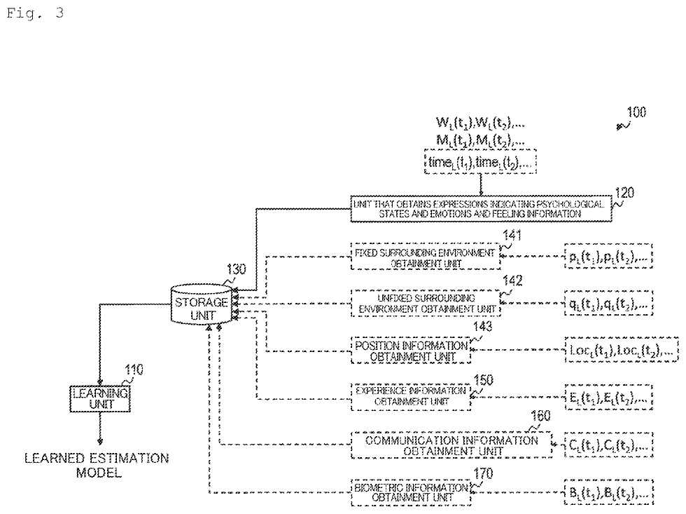

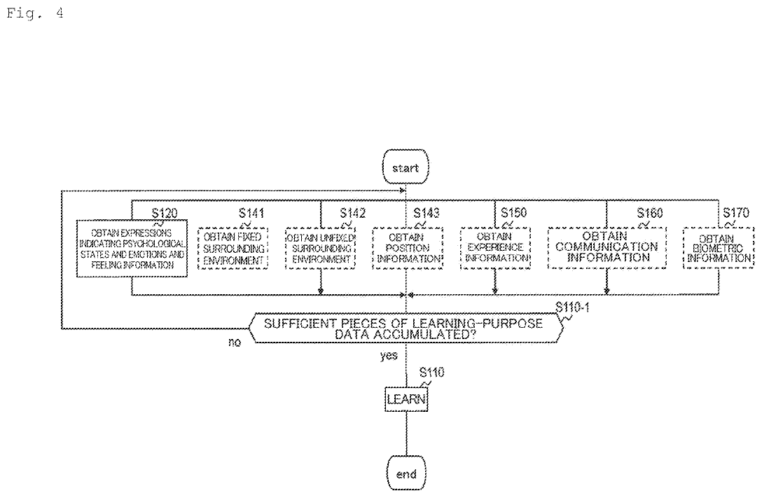

FIG. 3 shows a functional block diagram of the learning apparatus 100 according to the first embodiment, and FIG. 4 shows a processing flow thereof.

The learning apparatus 100 includes a learning unit 110, a unit 120 that obtains expressions indicating psychological states and emotions and feeling information, and a storage unit 130.

<Unit 120 that Obtains Expressions Indicating Psychological States and Emotions and Feeling Information, and Storage Unit 130>

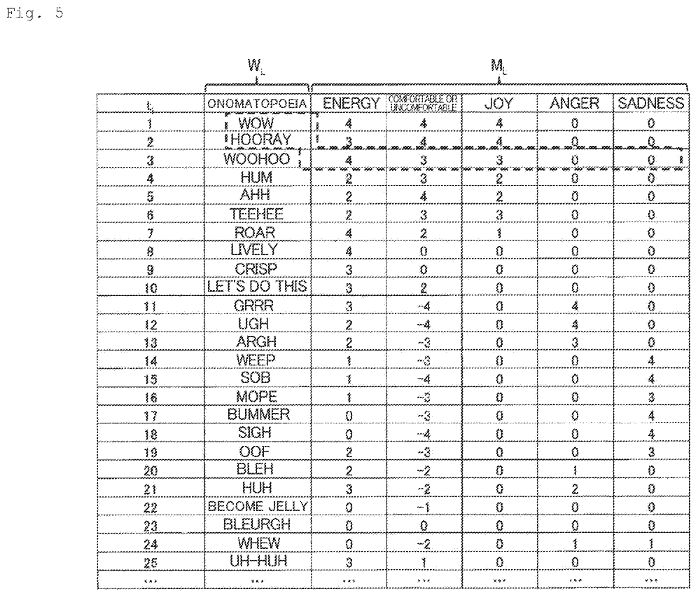

The unit 120 that obtains expressions indicating psychological states and emotions and feeling information accepts, from a user (an object person from which data is to be obtained), inputting of character strings of onomatopoeias that describe the states of the user himself/herself at the time of the input (learning-purpose expressions indicating psychological states and emotions) W_{L} (t₁), W_{L} (t₂), . . . , as well as feeling information indicating the feelings at that time (learning-purpose feeling information) M_{L} (t₁), M_{L} (t₂), . . . (S120), and stores the same into the storage unit 130. Thus, the learning-purpose expressions indicating psychological states and emotions W_{L} (t₁), W_{L} (t₂), . . . , as well as the learning-purpose feeling information M_{L} (t₁), M_{L} (t₂), . . . , are stored in the storage unit 130. FIG. 5 shows an example of data stored in the storage unit 130. Note, it is assumed that data is stored in the order of input performed by the user (that is to say, in the order of times at which the user performed input). In other words, it is assumed that data is stored. in the order accepted by the unit 120 that obtains expressions indicating psychological states and emotions and feeling information. In the example of FIG. 5, the indexes tᵢ indicating the order of input performed by the user (the order of acceptance by the unit 120 that obtains expressions indicating psychological states and emotions and feeling information) are stored together; however, in a case where the order of input performed by the user (the order of acceptance by the unit 120 that obtains expressions indicating psychological states and emotions and. feeling information) is known from the stored locations and the like, the indexes tᵢ need not be stored.

Feeling information indicates pre-set scales using a plurality of levels (9 levels or 5 levels in the above-mentioned example) as follows, for example.
(1) The extent of comfortableness or uncomfortableness as a feeling is represented in 9 levels, with 4 denoting the state of being comfortable, and −4 denoting the state of being uncomfortable.
(2) The extent of anger is represented in 5 levels, with 4 denoting the state of being angry, and 0 denoting the state of not being angry.
(3) The extent of energy is represented in 5 levels, with 4 denoting the state of being motivated, and 0 denoting the state of being unmotivated.

There may be one type of feeling information (e.g., one of the aforementioned (1) to (3)), or there may be a plurality of types of feeling information (e.g., the aforementioned (1) and (3), and the like).

For example, an input field for a character string of an onomatopoeia and an input field for feeling information are displayed on a display of a mobile terminal, a tablet terminal, and. the like, and the user inputs a character string of an onomatopoeia and feeling information via an input unit, such as a touchscreen.

Note that the input fields may be configured in. such a manner that character strings of predetermined types of onomatopoeias, as well as feeling information represented in a plurality of preset levels, are displayed for selection, yr may be configured to allow the user to perform input freely.

With regard to the timing of inputting of learning-purpose data, for example, a message that prompts inputting of a character string of an onomatopoeia and feeling information may be displayed to the user at an interval of a predetermined period via a display unit, such as a touchscreen, and the user may perform input in accordance with this message; also, the user may open an application that accepts inputting of a character string of an onomatopoeia and feeling information at an arbitrary timing and perform input.

<Learning Uni. 110>

Once the learning-purpose expressions indicating psychological states and emotions that are sufficient in amount for learning, as well as learning-purpose feeling information corresponding thereto, have been accumulated in the storage unit 130 (S110-1), the learning unit 110 retrieves learning-purpose expressions indicating psychological states and emotions and learning-purpose feeling information corresponding thereto from the storage unit 130, learns the estimation model (S110), and outputs the learned estimation model.

Note that as stated earlier, the estimation model is a model which. uses two or more chronological expressions indicating psychological states and emotions up to time (t), and which estimates a feeling after time (t). Note that time (t) denotes the time at which the t^{th} expression indicating a psychological state and an emotion was input. In the present embodiment, although the input time (acceptance time) is not obtained, the input order (acceptance order) is specified, and. thus it is possible to specify whether an expression indicating a psychological state and an emotion was input by time (t) at which the tth expression indicating a psychological state and an emotion was input, and whether feeling information is posterior to time (t).

For example, in the case of FIG. 2, the estimation model is a model which uses the (t−1)^{th} expression indicating a psychological state and. an emotion W (t−1), “ooph”, and the t^{th} expression indicating a psychological state and an emotion W (t) , “ow”, as inputs, and which estimates the (t+1)^{th} feeling information M (t+1). Thus, the learning apparatus 100 uses a pair of two or more chronological expressions indicating psychological states and emotions up to time (t) and learning-purpose feeling information indicating a feeling after time (t) as one pair of pieces of learning-purpose data (e.g., the portion enclosed by a dash line in FIG. 5), and learns the estimation model using a large number of pieces of learning-purpose data.

Note that the connection in temporal changes in a feeling is held with respect to an identical user. Therefore, even if a certain user ui has input the (t−1)^{th} expression indicating a psychological state and an emotion W (t'1), “ooph”, and another user u2 has input the t^{th} expression indicating a psychological state and an emotion W (t), “ow”, it is not possible to estimate which user will have what kind. of feeling at the time corresponding to (t+1). In view of this, the estimation model uses two or more chronological expressions indicating psychological states and emotions that were issued by a certain user by time (t) as inputs, and estimates the feeling of that user after time (t). Therefore, in a case where the unit 120 that obtains expressions indicating psychological states and emotions and feeling information obtains expressions indicating psychological states and emotions and. feeling information from a plurality of users, expressions indicating psychological states and emotions and feeling information. that were obtained from. each user are stored into the storage unit 130 together with a user-by-user identifier, and at the time of learning, learning is performed using a chronological series of expressions indicating psychological states and emotions and feeling information of each user. Note that the “issuance”of expressions indicating psychological states and emotions means conveyance of the expressions indicating psychological states and emotions to the outside by using. some form of. means, and is a concept that includes “inputting”of the expressions indicating psychological states and emotions via an input unit, such as a touchscreen, “speaking”of the expressions indicating psychological states and emotions, and so forth. Note that processing for a case where the expressions indicating psychological states and emotions are “spoken”will be described. later.

Note that learning-purpose data may be obtained from. one user. However, in a case where an unspecified number of object people serve as estimation. targets, it is desirable that learning-purpose data be obtained from a plurality. of users in order to be able to deal with the unspecified number of object people, and. also to obtain a sufficient number of pieces of learning-purpose data. That is to say, it is sufficient to have a chronological series of expressions indicating psychological states and emotions and feeling information on a user-by-user basis by preparing a large number of pairs of expressions indicating psychological states and emotions of a plurality of users and. feeling information at the time of the issuance of these expressions indicating psychological states and emotions, and use the chronological series as learning-purpose data. An estimation model that has been learned using such learning-purpose data is also referred to as a first estimation model. Furthermore, it is also permissible to consider an object person who serves as an estimation target of the estimation apparatus 200 as a new user (an object person from whom data is to be obtained), re-learn the first estimation model with use of learning-purpose data obtained from the new user, and output the re-learned estimation model as a model used in the estimation apparatus 200. Adopting such a configuration enables learning of an estimation model that takes the characteristics of an estimation target into consideration while obtaining a sufficient number of pieces of learning-purpose data.

FIG. 5 shows an example of a table composed of learning data. In this example, energy, joy, anger, and sadness are represented by numerical values in 5 levels, namely 0 to 4, and are evaluated to have a larger numerical value as the extent thereof increases. Comfortableness and uncomfortableness are represented by numerical values in nine levels, namely −4 to 4, and are evaluated to have a more positive value as the extent of “comfortableness”increases, and a more negative value as the extent of “uncomfortableness”increases.

### FIRST EXAMPLE OF ESTIMATION MODEL

An item (e.g., a table or a list) in which two or more chronological onomatopoeias (character strings) up to certain time are associated with feeling information after that time is used as an estimation model. For each piece of feeling information inside the table or the list, for example, a representative value (average value, median value, and the like) of feeling information that has been allocated by each person to an onomatopoeia included in learning-purpose data is used.

### SECOND EXAMPLE OF ESTIMATION MODEL

In this example, an estimation model is a model that has been learned through machine learning, such as a neural network, based on two or more chronological learning-purpose onomatopoeias up to certain time and on learning-purpose feeling information after that time. For example, a neural network which uses two or more chronological onomatopoeias (character strings) up to certain. time as inputs, and which outputs feeling information after that time, is used as the estimation model. In this case, the estimation model is learned as follows. Here, parameters of the neural network are updated repeatedly so that the result of estimation of feeling information obtained by inputting, to the neural network in which appropriate initial values have been. set in advance, two or more chronological onomatopoeias (character strings) by certain time that are included in learning-purpose data, becomes close to feeling information after that time that is included in the learning-purpose data. Note that when using learning-purpose data obtained by inputting a plurality of pieces of feeling information with respect to one onomatopoeia, learning may be performed so that the output of the estimation model also includes a list (set) of the plurality of pieces of feeling information.

In the foregoing manner, the learning apparatus 100 learns the estimation model. Next, the estimation apparatus will be described.

<Estimation Apparatus 200>

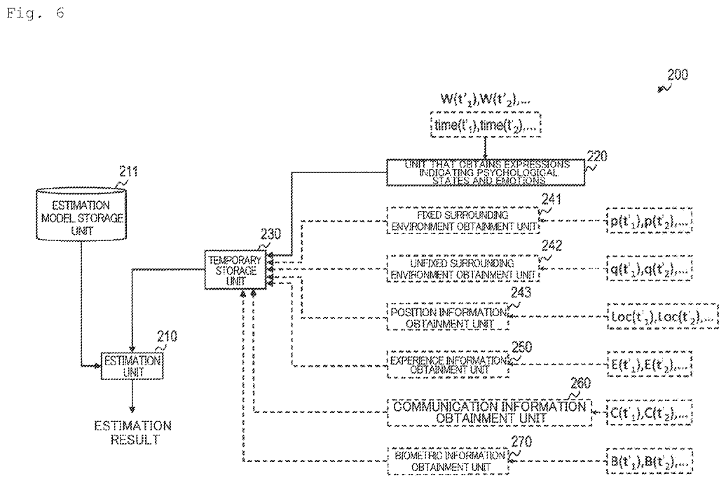

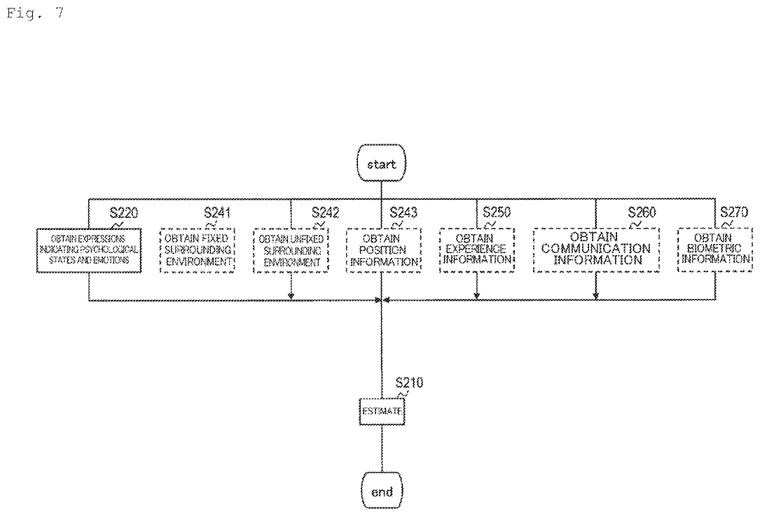

FIG. 6 shows a functional block diagram of the estimation apparatus 200 according to the first embodiment, and FIG. 7 shows a processing flow thereof.

The estimation apparatus 200 includes an estimation unit 210, an estimation model storage unit 211, a unit 220 that obtains expressions indicating psychological states and emotions, and a temporary storage unit 230.

<Unit 220 that Obtains Expressions Indicating Psychological States and Emotions, and Temporary Storage Unit 230>

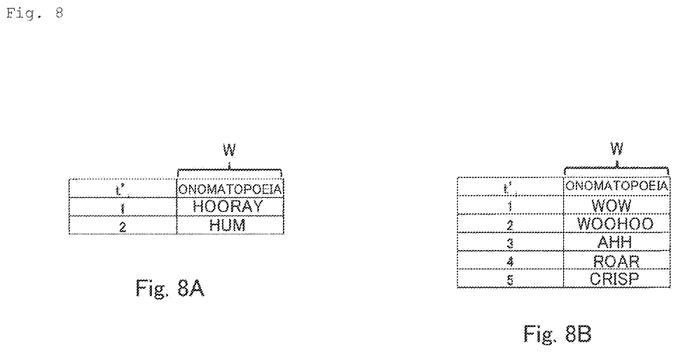

The unit 220 that obtains expressions indicating. psychological states and emotions accepts, from a user of the estimation. apparatus 200, inputting of character strings of onomatopoeias W (t′₁), W (t′₂), . . . , that describe the states of an object person at a plurality of times (t′₁), (t′₂), . . . (expressions indicating psychological states and emotions) (S220), and stores the character strings of onomatopoeias into the temporary storage unit 230. Note that the user of the estimation apparatus 200 (a person who estimates a feeling) and the object person (person whose feeling is to be estimated) may be an identical person (a person estimating a feeling of himself/herself by himself/herself), or may be different people. The temporary storage unit 230 stores the expressions indicating psychological states and emotions; FIG. 8 shows an example of data stored in the temporary storage unit 230. FIG. 8A shows an example of a case where inputting of expressions indicating psychological states and emotions W (t′₁), W (t′₂) at two times has been accepted, and FIG. 8B shows an example of a case where inputting of expressions indicating psychological states and emotions W (t′₁), . . . , W (t′₅) at five times has been accepted. Note, it is assumed that data is stored in the order of input performed by the user, that is to say, the order of acceptance by the unit 220 that obtains expressions indicating psychological states and emotions. Note that in the example of FIG. 8, the indexes t′ᵢ indicating the input order (acceptance order) are stored together; however, in a case where the input order (acceptance order) is known from the stored locations and the like, the indexes t′ᵢ need not be stored.

<Estimation Unit 210 and Estimation Model Storage Unit 211>

The learned estimation model that has been output from the learning apparatus 100 is stored in the estimation model storage unit 211 in advance. The estimation unit 210 retrieves two or more expressions indicating psychological states and emotions from the temporary storage unit 230. Then, using the learned estimation model that has been stored in the estimation model storage unit 211 in advance, the estimation unit 210 estimates the future feeling of the object person from the two or more expressions indicating psychological states and emotions of the object person and the input order (acceptance order) thereof (S210), and outputs the estimation result. Note that it is sufficient for the estimation unit 210 to retrieve, from the temporary storage unit 230, expressions indicating psychological states and emotions that are necessary for estimating the future feeling in the estimation model. Here, these necessary expressions indicating psychological states and emotions are specified by a learning method of the estimation model.

Furthermore, the estimation unit 210 may be configured to use a necessary estimation model depending on the purpose, that is to say, which feeling information is to be estimated. For example, (i) a learned estimation model that estimates “energy”, (ii) a learned estimation model that estimates “comfortableness and uncomfortableness”, (iii) a learned estimation model that estimates both “energy”and “comfortableness and uncomfortableness”, and the like may be prepared in the estimation model storage unit 211, and the estimation unit 210 may select a necessary estimation model in accordance with the purpose.

Note that it is sufficient for the estimation apparatus 200 to use two or more chronological expressions indicating psychological states and emotions up to time (t′) as inputs, and estimate a feeling after time (t′). It is sufficient that the estimation model be a model which uses two or more chronological expressions indicating psychological states and emotions up the time (t′), and which estimates a feeling after time (t′). Here, this estimation model is learned by the learning apparatus 100, and stored in the estimation model storage unit 211 of the estimation apparatus 200. For example, the number of chronological expressions indicating psychological states and emotions up to time (t′) that are used by the estimation apparatus 200 need not necessarily be two, and may be two or more. Furthermore, the order of issuance by the object person need not be consecutive. Similarly, the number of chronological expressions indicating psychological states and emotions up to time _{L}(t) that are used by the learning apparatus 100 need not necessarily be two, and may be two or more. In addition, the order of issuance by the user need not be consecutive. For example, the estimation apparatus 200 may estimate a feeling after time (t′) with use of the (t′−3)^{th}, (t′−1)^{th}, and the t′^{th} expressions indicating psychological states and emotions; in this case, it is sufficient that the estimation model learned by the learning apparatus 100 be a model that estimates a feeling after time (t) with use of the (t−3)^{th}, (t−1)^{th}, and the t^{th} expressions indicating psychological states and emotions. Furthermore, it is sufficient that a feeling estimated by the estimation apparatus 200 be a feeling after time (t+) corresponding to the t′^{th} expression indicating a psychological state and an emotion. For example, it is sufficient that the estimation model learned by the learning apparatus 100 be a model that estimates a feeling corresponding to the (t+2)^{th} and subsequent expressions indicating psychological states and emotions. In addition, the estimation apparatus 200 may estimate two or more feelings after time (t′). In this case, it is sufficient that the estimation model learned by the learning apparatus 100 be a model that estimates two or more feelings after time (t). For example, the estimation apparatus 200 may estimate the (t′+1)^{th} and (t′+2)^{th} feelings with use of the (t′−1)^{th} and t′^{th} expressions indicating psychological states and emotions. In this case, it is sufficient that the estimation model learned by the learning apparatus 100 be a model that estimates the (t+1)^{th} and (t+2)^{th} feelings with use of the (t−1)^{th} and t^{th} expressions indicating psychological states and emotions. These estimation model can be realized. depending on learning, and it is sufficient to set the inputs to and the outputs from the estimation models in consideration of the purpose of use, the cost, and the estimation accuracy of the estimation apparatus 200.

<Advantageous Effects>

With the foregoing configuration, a future feeling can be estimated based on expressions indicating psychological states and emotions up to the present time.

<First Exemplary Modification: Time>

The following description will be given with a focus on the differences from the first embodiment.

Here, it is presumed. that, because a feeling goes through temporal changes while maintaining a connection and there is also a connection between an expression indicating a psychological state and an emotion that was issued at a certain time point and a feeling at that time point, the use of these connections makes it possible to estimate, from a chronological series of expressions indicating psychological states and emotions that were input by certain time together with time information, a feeling at certain time after that time. The present exemplary modification is based on this presumption. For example, in FIG. 2, it is assumed that the feeling information M (t+1) at time (t+1) differs among a case where the interval between time (t−1) and time (t) is one minute, a case where the interval is one hour, and a case where the interval is one day. In other words, it is assumed. that the feeling information M (t+1) at time (t+1) differs depending on whether “ow”was input one minute, one hour, or one day after “ooph”was input. In view of this, in the present exemplary modification, an estimation model is learned by using the times corresponding to two or more expressions indicating psychological states and emotions as inputs. Then, in the present exemplary modification, with use of the estimation model obtained through this learning, a future feeling is estimated using the times corresponding to two or more expressions indicating psychological states and emotions as inputs.

<Unit 120 that Obtains Expressions Indicating Psychological States and Emotions and Feeling Information, and Storage Unit 130>

The unit 120 that obtains expressions indicating psychological states and emotions and feeling information in the learning apparatus 100 accepts, from a user, inputting of character strings of onomatopoeias that describe the states of the user himself/herself at the time of the input (learning purpose expressions indicating psychological states and emotions) W_{L} (t₁), W_{L} (t₂), . . . , as well as feeling information indicating the feelings at that time (learning-purpose feeling. information) M_{L} (t₁), M_{L} (t₂), . . . (S120) , obtains corresponding time _{L}(t₁), time _{L}(t₂), . . . , and stores pairs thereof into the storage unit 130 (see FIG. 3). Note that although the indexes tᵢ indicating the input order are not stored. into the storage unit 130 because the input order is known from the corresponding times, the indexes tᵢ indicating the input order may be stored into the storage unit 130.

The corresponding times may be the times at which the user input the character strings of onomatopoeias and feeling information (input times) via an input unit, such as a touchscreen, or may be the times at which the unit 120 that obtains expressions indicating psychological states and emotions and feeling information accepted the character strings of onomatopoeias and feeling information (acceptance times). In the case of the input times, it is permissible to adopt a configuration in which an input unit, such as a touchscreen, obtains the times from an internal clock, an NIP server, and the like and outputs the times to the unit 120 that obtains expressions indicating psychological states and emotions and feeling information; in the case of the acceptance times, it is permissible to adopt a configuration in which the unit 120 that obtains expressions indicating psychological states and emotions and feeling information obtains the acceptance times from, for example, an internal clock, an NTP server, and the like.

Note that the unit 120 that obtains expressions indicating psychological states and emotions and feeling information may be configured as follows. Specifically, in this configuration, a display unit, such as a touchscreen, displays a message that prompts inputting of the character strings of onomatopoeias and feeling information at preset time _{L}(t₁), time _{L}(t₂), . . . ; at each of the times of display, for example, at t₁, inputting of a character string of an onomatopoeia W_{L} (t₁) and feeling information M_{L} (t₁) at that time is accepted, and a pair of this input and corresponding time _{L}(t₁) is stored. into the storage unit 130, whereas at t₂ for example, inputting of a character string of an onomatopoeia W_{Lv} (t₂) and feeling information M_{L}(t₂) at that time is accepted, and a pair of this input and corresponding time _{L}(t₂) is stored into the storage unit 130.

<Learning Unit 110>

Once the learning-purpose expressions indicating psychological states and emotions that are sufficient in amount for learning, learning-purpose feeling information corresponding thereto, and the corresponding times have been accumulated in the storage unit 130 (S110-1), the learning unit 110 of the learning apparatus 100 retrieves learning-purpose expressions indicating psychological states and emotions, the times corresponding to the learning-purpose expressions indicating psychological states and emotions, and corresponding learning-purpose feeling information from the storage unit 130, learns the estimation model (S110), and outputs the learned estimation model. Note that the estimation model may be learned using corresponding time _{L}(t₁), time _{L}(t₂) , . . . as is. Also, the time periods that have elapsed since the issuance of expressions indicating psychological states and emotions before time _{L}(t₁), time _{L}(t₂), . . . (e.g., time _{L}(t₂)−time _{L}(t₁), _{L}(t₃)−time _{L}(t₂), . . . ) may be obtained, and the estimation model may be learned using the time periods that have elapsed since inputting of the previous expressions indicating psychological states and. emotions.

(First Example of Learning of Estimation Model)

For example, the learning apparatus 100 uses a set of two or more expressions indicating psychological states and emotions W_{L} (t), W_{L} (t−1), . . . up to certain. time _{L}(t), corresponding. time _{L}(t), time _{L}(t−1), . . . or the difference therebetween (time _{L}(t)−time _{L}(t−1), . . . ), and feeling information. M_{L} (t+1) after time _{L}(t) as one set of pieces of learning-purpose data, and learns the estimation model using a large number of pieces of learning-purpose data.

The estimation model according to the first example of learning of the present exemplary modification is a model which uses two or more chronological expressions indicating psychological states and. emotions up to time (t′), as well as the times corresponding to these expressions indicating psychological states and emotions or the difference between these times, as inputs, and which uses these inputs when estimating a feeling after time (t′) in the estimation apparatus 200.

(Second Example of Learning of Estimation Model)

Alternatively, for example, the learning apparatus 100 uses a set of two or more expressions indicating psychological states and emotions W_{L} (t), W_{L} (t−1), . . . up to certain time _{L}(t), the input order (acceptance order) t, t−1, . . . , a time interval |time _{L}(t)−time _{L}(t−1)|, . . . , and feeling information M_{L} (t+1) after time _{L}(t) as one set of pieces of learning-purpose data, and learns the estimation model using a large number of pieces of learning-purpose data.

The estimation model. according Co the second example of learning of the present exemplary modification is a model used by the estimation apparatus 200 in the following case. Specifically, This case refers to a case where the estimation apparatus 200 uses two or more chronological expressions indicating psychological states and emotions up to time (t′), the input order (acceptance order) of these expressions indicating psychological states and emotions, and the interval between. the times corresponding to these expressions indicating psychological states and emotions (time interval) as inputs, and estimates a feeling after time (t′).

<Unit 220 that Obtains Expressions Indicating Psychological States and Emotions, and Temporary Storage Unit 230>

The unit 220 that obtains expressions in psychological states and emotions of the estimation apparatus 200 accepts inputting of character strings of onomatopoeias W (t′₁), W (t′₂), . . . that describe the states of an object person at a plurality of times (expressions indicating psychological states and emotions) (S220), obtains corresponding time (t′₁), time (t′₂), . . . , and stores pairs thereof into the temporary storage unit 230. Thus, the expressions indicating psychological states and emotions, as well as corresponding time (t′₁), time (t′₂), . . . , are stored in the temporary storage unit 230. Note that although the indexes t′ᵢ indicating the input order (acceptance order) are not stored into the temporary storage unit 230 because the input order (acceptance order) is known from the corresponding times, the indexes t′ᵢ indicating the input order may be stored into the temporary storage unit 230.

<Estimation Unit 210 and Estimation Model Storage Unit 211>

The learned estimation model that has been output from the learning apparatus 100 of the present exemplary modification is stored in the estimation model storage unit 211 in advance. The estimation unit 210 of the estimation apparatus 200 retrieves two or more expressions indicating psychological states and emotions, as well as the times corresponding to the expressions indicating psychological states and. emotions, from the temporary storage unit 230.

(Exemplary Estimation for Case Where Estimation Model According to Aforementioned First Example of Learning is Used)

In a case where the estimation model according to the first example of learning of the present exemplary modification is used, the estimation unit 210 of the estimation apparatus 200 obtains the time difference from the corresponding times as necessary. Then, using the learned estimation model according to the first example of learning that has been stored in the estimation model storage unit 211 in advance, the estimation unit 210 estimates a future feeling of an object person from two or more expressions indicating psychological states and emotions of the object person and the times that respectively correspond to the expressions indicating psychological states and emotions, or the time difference therebetween (S210), and outputs the estimation result.

(Exemplary Estimation for Case Where Estimation. Model According to Aforementioned Second Example of Learning is Used)

In a case where the estimation. model according to the second example of learning of the present exemplary modification is used, the estimation unit 210 of the estimation apparatus 200 obtains the input order (acceptance order) and the time interval from the corresponding times. Then, using the learned. estimation model according to the second example of learning that has been stored in the estimation model storage unit 211 in advance, the estimation unit 210 estimates a future feeling of an object person from two or more expressions indicating psychological states and emotions of the object person, the input order (acceptance order) of the respective expressions indicating psychological states and emotions, and the time interval between the times that respectively correspond to the expressions indicating psychological states and emotions (S210), and outputs the estimation result.

Note that. in a case where the indexes t′ᵢ indicating the input order (acceptance order) are stored in the temporary storage unit 230, the indexes t′ᵢ indicating the input order (acceptance order) stored in the temporary storage unit 230 may be used as is without obtaining the input order (acceptance order) from the times.

With the foregoing configuration, the advantageous effects that are similar to the advantageous effects of the first embodiment can be achieved. Furthermore, a feeling can be estimated. more accurately by taking times into consideration.

<Second Exemplary Modification: Time>

The following description will be given with a focus on the differences from the first exemplary modification.

In the present exemplary modification, an estimation model is learned also using the times corresponding to feelings, and this learned estimation model is used also to estimate how much later the estimated future feeling will occur and to estimate the feeling at a designated future time,

<Learning Unit 110>

Once the learning-purpose expressions indicating psychological states and emotions that are sufficient in amount for learning, learning purpose feeling information corresponding thereto, and the corresponding times have been accumulated in the storage unit 130 (S110-1), the learning unit 110 of the learning apparatus 100 retrieves learning-purpose express ons indicating psychological states and emotions, learning-purpose feeling information. corresponding to the learning-purpose expressions indicating psychological states and emotions, the times corresponding to the learning-purpose express ons indicating psychological states and emotions, and the times corresponding to the learning-purpose feeling information from the storage unit 130, learns the estimation model (S110), and outputs the learned estimation model. For example, the learning apparatus 100 uses a set of two or more chronological expressions indicating psychological states and emotions up to time _{L}(t), the feeling at time _{L}(t+1) which is the time after time _{L}(t), and time _{L}(t) and time _{L}(t+1) or the difference therebetween time _{L}(t+1)—time _{L}(t) as one set of pieces of learning-purpose data, and learns the estimation model with use of a large number of pieces of learning-purpose data.

Note that the estimation model of the present exemplary modification is a model for which the estimation apparatus 200 uses two or more chronological expressions indicating psychological states and emotions up to time (t′) as inputs, and which is used by the estimation apparatus 200 in estimating a feeling after time (t′) and the time corresponding to The later feeling. Alternatively, the estimation model of the present exemplary modification is a model for which the estimation apparatus 200 uses two or more chronological expressions indicating psychological states and emotions up to time (t′) and a future time as inputs, and which is used by the estimation apparatus 200 in estimating a feeling at the future time.

<Estimation Unit 210 and Estimation Model Storage Unit 211>

The learned estimation model that has been output from the learning apparatus 100 of the present exemplary. modification is stored in the estimation model storage unit 211 in advance. The estimation unit 210 of the estimation apparatus 200 retrieves two or more expressions indicating psychological states and. emotions W (t′), W (t′1), . . . and corresponding times (t′) from the temporary storage unit 230, estimates a future feeling of an object person and the time corresponding this feeling from the two or more expressions indicating psychological states and emotions or the object person with use of the learned estimation model that has been stored in the estimation model storage unit 211 in advance (S210), and outputs the estimation result. That is to say, how much later in the future the feeling will occur is output together with the result of estimating the feeling. Alternatively, the estimation unit 210 may include a non-illustrated input unit and accept inputting of a future time, that is to say, designation of how much later a future feeling to be obtained as the estimation result will occur, a user of The estimation apparatus 200 designates how much later the future feeling to be obtained by the estimation apparatus 200 as the estimation result will occur, and the estimation unit 210 estimates a future feeling in accordance with the designated content.

With the foregoing configuration, the advantageous effects that are similar to the advantageous effects of the first embodiment can be achieved. Furthermore, it is possible to take into consideration how much later a future feeling will occur from time (t′) corresponding to an expression indicating a psychological state and an emotion P (t′).

Note that the present exemplary modification and the first exemplary modification may be combined. An estimation model according to a combination of the present exemplary modification and the first exemplary modification is, for example, the following models.

(First Exemplary Combination)

An estimation model is a model which uses two or more chronological expressions indicating psychological states and emotions up to time (t′), as well as the times corresponding to these expressions indicating psychological states and emotions or the time difference therebetween, as inputs, and which estimates a feeling after time (t′) and the time corresponding to the later feeling.

(Second Exemplary Combination)

An estimation model is a model which uses two or more chronological expressions indicating psychological states and emotions up to time (t′), the times corresponding to these expressions indicating psychological states and emotions or the time difference therebetween, and a future time as inputs, and which estimates a feeling a the future time.

(Third Exemplary Combination)

An estimation model is a model which. uses two or more chronological expressions indicating psychological states and emotions up to time (t′), the input order (acceptance order) of these expressions indicating psychological states and. emotions, and the interval (time interval) between the times corresponding to these expressions indicating psychological states and emotions as inputs, and which estimates a feeling after time (t′) and the time corresponding to the later feeling.

(Fourth Exemplary Combination)

An estimation model is a model which uses two or more chronological expressions indicating psychological states and emotions up to time (t′), the input order (acceptance order) of these expressions indicating psychological states and emotions, the interval (time interval) between. the times corresponding to these expressions indicating psychological state and emotions, and a future time as inputs, and which estimates a feeling at the future time.

In the estimation apparatus 200 according to a combination of the present exemplary modification and the first exemplary modification, one of the foregoing estimation models is stored in the estimation model storage unit 211 in advance, and the estimation unit 210 obtains and outputs the future feeling of an object person and the time corresponding to this feeling, or the feeling of the object person at the designated future time, as the estimation result.

<Third Exemplary Modification: Other Information>

The accuracy of estimation of a feeling after certain time can be increased by taking into consideration not only two or more expressions indicating psychological states and emotions up to the certain time, but also other information up to the certain time. For example, possible examples of other information. include fixed surrounding environment information, unfixed surrounding environment information, position information, experience information, communication information, biometric information, and other types of information that influence a later feeling. An estimation model is learned by providing these pieces of information in addition to two or more expressions indicating psychological st&tes and emotions and feeling information, and a feeling is estimated by providing these pieces of information to the two or more expressions indicating psychological states and emotions with use of the estimation model obtained through this learning.

<Learning Apparatus 100>

The learning apparatus 100 includes not only the learning unit 110, the unit 120 that obtains expressions indicating psychological states and emotions and feeling information, and the storage unit 130, but also at least one of a fixed surrounding environment obtainment unit 141, an unfixed. surrounding environment obtainment unit 142, a position information obtainment unit 143, an experience information obtainment unit 150, a communication. information obtainment unit 160, and a biometric information obtainment unit 170 (see FIG. 3).

<Estimation Apparatus 200>

The estimation apparatus 200 includes riot only the estimation an it 210, the unit 220 that obtains expressions indicating psychological states and emotions, and the temporary storage unit 230, but also at least one of a fixed surrounding environment obtainment unit 241, an unfixed surrounding environment obtainment unit 242, a position information obtainment unit 243, an experience information obtainment an it 250, a communication information obtainment unit 260, and a biometric information obtainment unit 270 (see FIG. 6).

<Fixed Surrounding Environment Obtainment Units 141 and 241>

The fixed surrounding environment obtainment unit 141 obtains information p_{L}(t) related to a fixed surrounding environment associated with a location (S141), and stores the information into the temporary storage unit 130.

Similarly, the fixed surrounding environment obtainment unit 241 obtains information p(t′) related to a fixed surrounding environment associated with a location (S241), and stores the information into the temporary storage unit 230.

For example, information related to a fixed surrounding environment associated with a location may be under such categories as “eating and drinking facility”and “play facility,”or may be, for example, a unique name of a lower level, such as “oo Amusement Park”and “xx Zoo”. For example, the estimation apparatus 200 learns an estimation model based on the presumption that a feeling at. time (t+1) is influenced in a case where the surrounding environment has changed between time (t−10) to time (t), or that a feeling at time (t+1) is influenced by the difference between the surrounding environment at time (t−1) and the surrounding environment at time (t). Then, the estimation apparatus 200 estimates a feeling with use of the estimation model that has been obtained through this learning. For example, using onomatopoeias that were input before and after entering a certain facility and information related to two fixed surrounding environments indicating the existence or non-existence inside this facility, a later feeling is estimated.

For example, the fixed surrounding environment obtainment units 141 and 241 have a PPS function and a database in which position information is associated with fixed surrounding environments, obtain the position information with use of the GPS function, and obtain information related to the fixed surrounding environments associated with the position information from the database. Furthermore, a user of the learning apparatus 100 and a user of the estimation apparatus 200 may input the same, similarly to the unit 120 that obtains expressions indicating psychological states and emotions and feeling information and the unit 220 that obtains expressions indicating psychological states and emotions.

<Unfixed Surrounding Environment Obtainment Units 142 and 242>

The unfixed surrounding environment obtainment unit 142 obtains information q_{L}(t) which is not associated with a location and which is related to an unfixed surrounding environment (S142), and stores the information into the temporary storage unit 130.

Similarly, the unfixed surrounding environment obtainment unit 242 obtains information q (t′) which is riot associated with a location and which is related to an unfixed surrounding environment (S242), and stores the information into the temporary storage unit 230.

For instance, possible examples of information which is not associated with a location and which is related to an unfixed surrounding environment include meteorological information, such as the air temperature, humidity, and the amount of rain. For example, the estimation apparatus 200 learns an estimation model based on the presumption that feeling at time (t+1) is influenced in a case where the surrounding environment has changed between time (t−1) to time (t), or that a feeling at time (t+1) is influenced by the difference between the surrounding environment at time (t−1) and the surrounding environment at time (t). Then, the estimation apparatus 200 estimates a feeling with use of the estimation model that has been obtained through this learning For example, using onomatopoeias that were input before and after rainfall and information related to two unfixed surrounding environments indicating whether there has been rainfall (e.g., the amount of rain), a later feeling is estimated.

For example, the unfixed surrounding environment obtainment units 142 and. 242 have a P08 function and an information collection function, obtain position information with use of the GPS function, and obtain meteorological information and the like corresponding to the position information from, for example, a meteorological observatory and the like with use of the information collection function. Furthermore, the unfixed surrounding environment obtainment units 142 and 242 may include a sensor that obtains, for example, such meteorological information as the air temperature, and obtain the meteorological information and the like. Furthermore, a user of the learning apparatus 100 and a user of the estimation apparatus 200 may input the same, similarly to the unit 120 that obtains expressions indicating psychological states and emotions and feeling information and the unit 220 that obtains expressions indicating psychological states and emotions.

<Position Information Obtainment Units 143 and 243>

The position information obtainment unit 143 obtains position information Loc_{L}(t) itself (S143), and stores the same into the storage unit 130.

Similarly, the position information obtainment unit. 243 obtains position information Loc (t′) itself (S243), and stores the same into the temporary storage unit 230.

For example, an estimation model is learned based on the presumption that a feeling at time (t+1) is influenced in a case where the location has changed between. time (t−1) to time (t), or that a feeling at time (t+1) is influenced. by the difference between the location at time (t−1) and the location at time (t) or by the extent of that difference (e.g., the distance traveled), and a feeling is estimated using the estimation model obtained through this learning. For example, using onomatopoeias that were input before and after the travel and two pieces of position information indicating whether the travel was made and the distance traveled, a later feeling is estimated.

For example, the position information obtainment units 143 and 243 have a GPS function, and obtain the pieces of position information. Furthermore, a user of the learning apparatus 100 and a user of the estimation apparatus 200 may input the same, similarly to the unit 120 that obtains expressions indicating psychological states and emotions and feeling information and the unit 220 that obtains expressions indicating psychological states and emotions.

<Experience Information Obtainment Units 150 and 250>

The experience information obtainment unit 150 obtains experience information E_{L}(t) related to an experience of a user (S150), and stores the same into the storage unit 130,

Similarly, the experience information obtainment unit 250 obtains experience information (t′) related to an experience of an object person (S250), and stores the same into the temporary storage unit 230.

For instance, possible examples of the experience information include information indicating whether there have been an experience of eating a certain food, an experience of listening to a certain piece of music, and an experience of playing a certain game. For example, the estimation apparatus 200 learns an estimation model based on the presumption that a feeling at time (t+1) is influenced in a case where the experience information has changed between time (t−1) to time (t), or that a feeling at time (t+1) is influenced by the difference between the experience information at time (t−1) and the experience information at time (t). Then, the estimation apparatus 200 estimates a feeling with use of the estimation model that has been obtained through this learning. For example, using onomatopoeias that were input before and after a live music concert and two pieces of experience information indicating whether there has been an experience of going to a live concert, a later feeling is estimated.

For example, the experience information obtainment units 150 and 250 have a GTS function and a database in which position information is associated with facilities that provide predetermined experiences (e.g., restaurants, live concert venues, and attraction facilities), obtain the position information with use of the GPS function, and obtain information indicating the predetermined experiences provided in the facilities associated with the position information from the database. Furthermore, a user of the learning apparatus 100 and a user of the estimation apparatus 200 may input the same, similarly to the unit 120 that obtains expressions indicating psychological states and emotions and feeling information and the unit 220 that obtains expressions indicating psychological states and emotions.

<Communication Information Obtainment Units 160, 260>

The communication information obtainment unit 160 obtains communication information C_{L}(t) related to communication of a user (S160), and stores the same into The storage unit 130.

Similarly, the communication information obtainment unit 260 obtains communication information C (t′) related. to communication of an object person (S260), and stores the same into the temporary storage unit 230.

For instance, possible examples of the communication information include information indicating a person who was met, the facial expression of the user or object person himself/herself, and the facial expression of the person who was met. For example, the estimation apparatus 200 learns an estimation model based on the presumption that a feeling at time (t+1) is influenced in a case where the communication information has changed between time (t−1) to time (t), or that a feeling at time (t+1) is influenced by the difference between the communication information at time (t−1) and the communication information at time (t). Then, the estimation apparatus 200 estimates a feeling with use of the estimation model that has been obtained through this learning. For example, using onomatopoeias that were input before and after meeting a friend and two pieces of communication information indicating the facial expressions of the user or object person himself/herself, a later feeling is estimated.

For example, the communication information obtainment units 160 and 260 have a shooting function, a facial authentication function, and a facial expression detection function, perform facial authentication with respect to a person who was shot using the shooting function, obtain information indicating a person who has been met, detect the facial expression of the person. who has been met or the object person with use of the facial expression detection function, and obtain information indicating the facial expression. Furthermore, in a case where there is, for example, a function that enables an object person and a person who has been met by the object person to exchange information indicating the identities thereof with each other, information indicating the person who has been met may be obtained using this function. Furthermore, a user of the learning apparatus 100 and a user of the estimation apparatus 200 may input the same, similarly to the unit 120 that obtains expressions indicating psychological states and emotions and feeling information and. the unit 220 that obtains expressions indicating psychological states and emotions.

<Biometric Information Obtainment Units 170 and 270>

The biometric information obtainment unit 170 obtains biometric information (t) of a user (S170), and stores the same into the storage unit 130.

Similarly, the biometric information obtainment unit 270 obtains biometric information B (t′) of an object person (S270), and stores the same into the temporary storage unit 230.

For instance, possible examples of biometric information include information indicating a heart rate, breathing, and facial expression. For example, the estimation apparatus 200 learns an estimation model based on the presumption that a feeling at time (t+1) is influenced in a case where the biometric information has changed between time (t−1) to time (t), or that a feeling at time (t+1) is influenced by the difference between the biometric information at time (t−1) and the biometric information at time (t). Then, the estimation apparatus 200 estimates a feeling. For example, a feeling is estimated from a change in a heart rate or breathing. Furthermore, a feeling is estimated by learning, for example, what kind of influence is exerted on a feeling at time (t+1) in a case where a heart rate or breathing has changed, or has not changed, when such onomatopoeias as “thump”have been obtained,

For example, the biometric information obtainment units 170 and 270 have a function of obtaining biometric information, and obtain the biometric information, The biometric information obtainment units 170 and 270 include, for example, an application compatible with a wearable device, such as hitoe®, and obtain biometric information of an object person.

<Learning Unit 110>

Once the learning-purpose expressions indicating psychological states and emotions that are sufficient in amount for learning, learning-purpose feeling information corresponding thereto, and the following (i) to (vi) have been accumulated in the storage unit 130 (S110-1), the learning unit 110 retrieves learning-purpose expressions indicating psychological states and emotions, learning-purpose feeling information corresponding thereto, and (i) to (vi) from the storage unit 130, learns the estimation model (S110), and outputs the learned estimation model.

(i) Information related to a fixed surrounding environment of a person who input an expression indicating a psychological state and an emotion at the time of the input, which is associated with a location

(ii) Information related to an unfixed surrounding environment of a person who input an expression indicating a psychological state and an emotion at the time or the input, which is not associated with a location

(iii) position information of a person who input an expression indicating a psychological state and an emotion at the time of the input

(iv) experience information related to an experience of a person who input an expression indicating a psychological state and an emotion at the time of the input

(v) communication information related to communication of a person who input an expression indicating a psychological state and an emotion at the time of the input

(vi) biometric information or a person who input an expression indicating a psychological state and an emotion at the time of the input

Note that there is no need to perform learning with use of all of (i) to (vi), and it is sufficient to obtain and store information necessary for the estimation and perform learning based thereon. It is sufficient to use a chronological series of at least one of (i) to (vi).

The estimation model of the present exemplary modification is a model for which the estimation apparatus 200 uses two or more chronological expressions indicating psychological states and. emotions up to time (t′) and a chronological series of at least one of (i) to (vi) corresponding to these expressions indicating psychological states and emotions as inputs, and which is used by the estimation apparatus 200 in estimating a feeling after time (t′)

<Estimation Unit 210 and Estimation Model Storage Unit 211>

The learned estimation model that has been output from the learning apparatus 100 of the present exemplary modification is stored in the estimation model storage unit 211 in advance. The estimation unit 210 retrieves two or more expressions indicating psychological states and emotions and at least one of the aforementioned (i) to (vi), which has been used in learning in the learning unit 110, from the temporary storage unit 230, estimates a future feeling from the two or more expressions indicating psychological states and emotions and at least one of (i) to (vi) with use of the learned estimation model that has been stored in the estimation model storage unit 211 in advance (S210), and outputs the estimation result.

<Advantageous Effects>

With the foregoing configuration, the advantageous effects that are similar to the advantageous effects of the first embodiment can be achieved. Furthermore, a feeling can be estimated more accurately by taking at least one of (i) to (vi) into consideration. Note that the present exemplary modification and the first and second exemplary modifications may be combined.

Note that although the present exemplary modification has been described under the assumption that the timing at which the fixed surrounding environment obtainment unit, the unfixed surrounding environment obtainment unit, the position information obtainment unit, the experience information obtainment unit, the communication information obtainment unit, and. the biometric information obtainment unit obtain respective pieces of information is the same as the timing at which the unit 220 obtains expressions indicating psychological states and emotions obtains expressions indicating psychological states and emotions, different obtainment units may perform the obtainment at different timings. It is permissible to use respective pieces of information at a timing that is closest to the timing of the obtainment of expressions indicating psychological states and emotions, supplement a lack of information, or thin out excess pieces of information.

<Fourth Exemplary Modification

Although the first embodiment has been described under the assumption that a user of the learning apparatus 100 and a user of the estimation apparatus 200 input character strings of onomatopoeias, no limitation is intended by inputting of the character string itself.

For example, an illustration, an image, or the like that is in one-to-one association with an onomatopoeia may be input. In this case, it is permissible to provide a database in which onomatopoeias and illustrations, images, or the like are associated, use an illustration, an image, or the like as an input, and retrieve a character string of an onomatopoeia corresponding thereto from the database.

Also, inputting of a character string of an onomatopoeia may be accepted by, for example automatically extracting the character string of the onomatopoeia included. in the result performing sound recognition with respect to a speech made by an object person. For example, it is permissible to use a sound signal as an input instead of a character string of an onomatopoeia, obtain the result of sound recognition by performing sound recognition processing in a non-illustrated sound recognition unit, extract a character string of an onomatopoeia from the result, and output the extracted character string. For example, a database that has stored target character strings of onomatopoeias is provided, and a character string of an onomatopoenia is extracted from the result of sound recognition with reference to this database.

In addition, in an estimation phase, it is permissible to use, as an input, a character string of an onomatopoeia that has been automatically extracted from a character string of text input when, for example, an object person creates a mail or creates a comment to be posted on the web, and to use, as an input, a character string of an onomatopoeia that has been automatically extracted from the result of performing sound recognition with respect to a voice of an object person when the object person talks on a mobile telephone and the like.

Moreover, in a learning phase, learning can be performed with use of chronological items which were issued by the same person (a character string of text input when creating a mail or creating a comment to be posted on the web, or the result of sound recognition) and which include both of an onomatopoeia and a word related to a feeling, whether that person is an object person or not, as long as the items are chronological. At this time, “words related to feelings”that have been obtained beforehand through a research, learning that has been performed separately, and the like may be Generally associated with feeling information in advance, a character string of a word related to a feeling may be automatically extracted similarly to the case of onomatopoeias, and the result of converting the extracted word related to the feeling into feeling information based on the aforementioned association may be used as an input.

Note that the present exemplary modification and the first to third exemplary modifications may be combined.

<Fifth Exemplary Modification>

Although the unit 120 that obtains expressions indicating psychological states and. emotions and feeling information of the learning apparatus 100 uses learning-purpose expressions indicating psychological states and emotions W_{L} (t₁), W_{L} (t₂), . . . , as well as learning-purpose feeling information M_{L} (t₁), M_{L} (t₂), . . . , as inputs in the present embodiment, it is permissible to adopt a configuration in which only learning-purpose expression s indicating psychological states and emotions W_{L} (t₁), W_{L} (t₂), . . . are used as inputs, and learning-purpose feeling information M_{L} (t₁), M_{L} (t₂), . . . corresponding to the expressions indicating psychological states and emotions W_{L} (t₁), W_{L} (t₂), . . . are obtained using the method of NPL 1.

Note that the present exemplary modification and the first to fourth exemplary modifications may be combined.

<Other Exemplary Modifications>

The present invention is not limited to the above-described embodiment and exemplary modifications. For example, various types of processing that have been described above are not limited to being executed chronologically in accordance with the description, and may be executed in parallel, or individually, depending on the processing capability of an apparatus that executes the processing, or as necessary. In addition, changes can be made as appropriate within a range that does not depart from the intent of the present invention

<Program and Recording Medium>

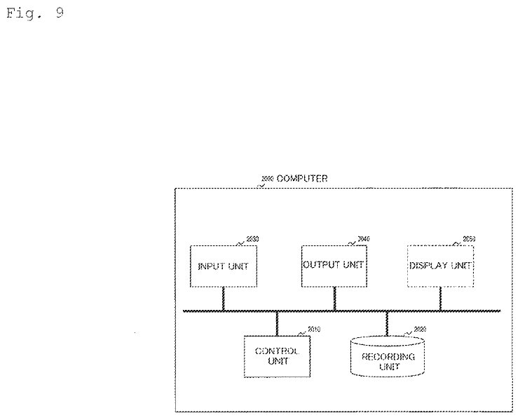

Various types of processing that have been described above can be carried out by causing a recording unit 2020 of a computer shown in FIG. 9 to read in a program that realizes the execution of each step of the above-described method, and to make a control unit 2010, an input unit 2030, an output unit 2040, and the like operate.

The program that describes the contents of such processing can be recorded on a recording medium that can be read by the computer. Any item, such as a magnetic recording apparatus, an optical disc, a magneto-optical recording medium, a semiconductor memory, and so forth, may be used as the recording medium that can be read by the computer.

Also, this program is distributed by, for example, sales, assignment, lease, etc. of a portable recording medium, such as a DVD and a CD-ROM, on which this program is recorded. Furthermore, it is permissible to adopt a configuration in which this program is stored in a storage apparatus of a server computer, and this program is distributed by transferring this program from the server computer to another computer via a network.

The computer that executes such a program, for example, first stores the program recorded on the portable recording medium, or the program transferred from the server computer, into a storage apparatus thereof. Then, at the time of the execution of processing, this computer reads the program stored in a recording medium thereof, and executes processing in accordance with the read program. Also, in another mode for executing this program, the computer may read. the program directly from the portable recording medium and execute processing in accordance with this program, and furthermore, each time the program is transferred from the server computer to this computer, processing may be executed sequentially in accordance with the received program in addition, it is permissible to adopt a configuration in which the program is riot transferred from the server computer to this computer, and the above-described processing is executed by a so-called ASP (Application Service Provider) service that realizes the functions of processing only via an instruction for the execution thereof and. the obtainment of the result. Note, it is assumed that the program according to the present mode includes information which is to be provided for use in processing performed by an electronic computational device, and which is similar to the program (e.g., data which does not represent a direct command to the computer, but which has a property that defines processing of the computer).

Furthermore, although it is assumed in the present mode that. the present apparatus is configured by causing a predetermined program to be executed on the computer, at least a part of the contents of processing may be realized in the form of hardware.

1. A learning apparatus, comprising:
a storage unit that stores at least a learning-purpose expression indicating a psychological state and an emotion, and learning-purpose feeling information indicating a feeling at the time of issuance of the learning-purpose expression indicating the psychological state and the emotion; and
a learning unit that learns an-estimation model, using a plurality of pieces of learning data, a piece of learning data being a set including at least a chronological series of two or more expressions indicating psychological states and emotions up to time (t) and learning-purpose feeling information indicating a feeling after the time (t), the estimation model estimating a feeling after a certain time by using the chronological series of two or more expressions indicating psychological states and emotions up to the certain time as an input.

2. An estimation apparatus, comprising
an estimation unit that uses at least a chronological series of two or more expressions indicating psychological states and emotions up to a certain time as an input, and of an estimation model that estimates a feeling after the certain time to estimate a future feeling of an object person based on at least input two or more expressions indicating psychological states and emotions of the object person and an input order thereof.

3. The estimation apparatus according to claim 2, wherein
the estimation model estimates the feeling after the certain time by at least one of:
using at least a time corresponding to the two or more expressions indicating psychological states and emotions up to the certain time as the input, or by also
using an acceptance order of the two or more expressions indicating psychological states and emotions up to the certain time and a time interval between the times corresponding to the expressions indicating psychological states and emotions as the input, and
wherein the estimation unit estimates the future feeling of the object person based on the times corresponding to the inputted two or more expressions indicating psychological states and emotions, or on the acceptance order of the inputted two or more expressions indicating psychological states and emotions and the time interval between the times corresponding to the expressions indicating psychological states and emotions.

4. The estimation apparatus according to claim 2, wherein
the estimation model is a model that estimates the feeling after the certain time by also using, as an additional input, at least one of:
information that is associated with a location and is related to a fixed surrounding environment,
information that is not associated with a location and is related to an unfixed surrounding environment,
position information,
experience information related to an experience,
communication information related to a communication, or
biometric information, and
wherein the estimation unit further estimates the future feeling of the object person based on at least one of:
first information of the object person indicating a psychological state and an emotion at the time of the input, the first information being associated with a location and being related to a fixed surrounding environment,
second information of the object person indicating a psychological state and an emotion at the time of the input, wherein the second information is not associated with a location and being is related to an unfixed surrounding environment,
position information of the object person indicating a psychological state and an emotion at the time of the input,
experience information related to an experience of the object person indicating a psychological state and an emotion at the time of the input,
communication information related to communication of the object person indicating a psychological state and an emotion at the time of the input, and
biometric information of the object person indicating a psychological state and an emotion at the time of the input.

5. The estimation apparatus according to claim 4, wherein the estimation model is learned by the learning apparatus comprising:
a storage unit that stores at least a learning-purpose expression indicating a psychological state and an emotion, and learning-purpose feeling information indicating a feeling at the time of issuance of the learning-purpose expression indicating the psychological state and the emotion; and
a learning unit that learns an-estimation model, using a plurality of pieces of learning data, a piece of learning data being a set including at least a chronological series of two or more expressions indicating psychological states and emotions up to time (t) and learning-purpose feeling information indicating a feeling after the time (t), the estimation model estimating a feeling after a certain time by using the chronological series of two or more expressions indicating psychological states and emotions up to the certain time as an input.

6. A method, wherein a storage unit stores at least a learning-purpose expression indicating a psychological state and an emotion, and learning-purpose feeling information indicating a feeling at the time of issuance of the learning-purpose expression indicating the psychological state and the emotion, the method comprising:
learning an estimation model, using a plurality of pieces of learning data, one piece of the learning data being a set including at least a chronological series of two or more expressions indicating psychological states and emotions up to time (t) and learning-purpose feeling information indicating a feeling after the time (t), the estimation model estimating a feeling after a certain time is learned by using at least a chronological series of two or more expressions indicating psychological states and emotions up to the certain time as an input.

7. The method of claim 6, further comprising
an estimation step of estimating, using at least a chronological series of two or more expressions indicating psychological states and emotions up to a certain time as an input, and with use of an estimation model that estimates a feeling after the certain time, a future feeling of an object person based on at least input two or more expressions indicating psychological states and emotions of the object person and an input order thereof.

8. (canceled)

9. The estimation apparatus according to claim 3, wherein
the estimation model is a model that estimates the feeling after the certain time by also using, as an additional input, at least one of:
information that is associated with a location and is related to a fixed surrounding environment,
information that is not associated with a location and is related to an unfixed surrounding environment,
position information,
experience information related to an experience,
communication information related to a communication, or biometric information, and
wherein the estimation unit further estimates the future feeling of the object person based on at least one of:
first information of the object person indicating a psychological state and an emotion at the time of the input, the first information being associated with a location and being related to a fixed surrounding environment,
second information of the object person indicating a psychological state and an emotion at the time of the input, wherein the second information is not associated with a location and is related to an unfixed surrounding environment,
position information of the object person indicating a psychological state and an emotion at the time of the input,
experience information related to an experience of the object person indicating a psychological state and an emotion at the time of the input,
communication information related to communication of the object person indicating a psychological state and an emotion at the time of the input, and
biometric information of the object person indicating a psychological state and an emotion at the time of the input.

10. The estimation apparatus according to claim 2, wherein the estimation model is learned by the learning apparatus comprising:
a storage unit that stores at least a learning-purpose expression indicating a psychological state and an emotion, and learning-purpose feeling information indicating a feeling at the time of issuance of the learning-purpose expression indicating the psychological state and the emotion; and
a learning unit that learns an estimation model, using a plurality of pieces of learning data, a piece of learning data being a set including at least a chronological series of two or more expressions indicating psychological states and emotions up to time (t) and learning-purpose feeling information indicating a feeling after the time (t), the estimation model estimating a feeling after a certain time by using the chronological series of two or more expressions indicating psychological states and emotions up to the certain time as an input.

11. The estimation apparatus according to claim 3, wherein the estimation model is learned by the learning apparatus comprising:
a storage unit that stores at least a learning-purpose expression indicating a psychological state and an emotion, and learning-purpose feeling information indicating a feeling at the time of issuance of the learning-purpose expression indicating the psychological state and the emotion; and
a learning unit that learns an estimation model, using a plurality of pieces of learning data, a piece of learning data being a set including at least a chronological series of two or more expressions indicating psychological states and emotions up to time (t) and learning-purpose feeling information indicating a feeling after the time (t), the estimation model estimating a feeling after a certain time by using the chronological series of two or more expressions indicating psychological states and emotions up to the certain time as an input.
